(19) United States
(12) Patent Application Publication
(10) Pub. No.: US 2005/0081857 A1
(43) Pub. Date: Apr. 21, 2005

(54) Multiple filament nasal strip with high peel angle release

(76) Inventor: Gustav Fenton, Minneapolis, MN (US)

Correspondence Address: John F. Klos;Fulbright & Jaworski L.L.P., 2100 IDS Center, 80 South Eighth Street, Minneapolis, MN, 55402-2112, US

(21) Appl. No.: 10/925,278

(22) Filed: Aug. 23, 2004

Related U.S. Application Data
(60) Provisional application No. 60/497,211, filed on Aug. 21, 2003.

(51) Int. Cl. A62B 7/00; A62B 9/00; A61M 15/00

(52) U.S. Cl. 128/206.12; 128/206.14

(57) ABSTRACT

The present invention relates to improved nasal dilators that prevent the outer wall of tissue of nasal passages of a nose from drawing in during breathing. The improved nasal dilator has a resilient element, or “spring,” made up of a plurality of small filaments for keeping the nasal passages from drawing in while breathing. The filaments may be a variety of shapes and sizes and may run both along the length of the nasal dilator and at a variety of different angles relative to the length of the nasal dilator. The filaments further allow the nasal dilator of the present invention to be removed from the nose in a “top-to-bottom” fashion. The top-to-bottom peel method allows a greater peel angle and so results in less peel force being transferred to the skin of the use.

This application claims priority to U.S. Provisional Patent Application No. 60/497,211, filed on Aug. 21, 2003.

### FIELD OF THE INVENTION

The invention relates to nasal dilators for preventing the outer wall tissue of the nasal passages of a nose from drawing in during breathing. More particularly, the invention provides an apparatus and method of use for a nasal dilator that has a resilient spring force resulting from multiple filaments.

### BACKGROUND OF THE INVENTION

Nasal dilators are well known. For example, U.S. Pat. Nos. 5,533,499, 5,533,503 and 6,318,362, each of which are herein incorporated by reference, disclose nasal dilators. These nasal dilators comprise a truss member having a first end region adapted to engage the outer wall tissue of a first nasal passage and a second end region of the truss member is configured to engage the outer wall tissue of a second nasal passage. The first and second end regions of the truss member are coupled to one another by an intermediate segment. The intermediate segment is configured to traverse a portion of the nose located between the first and second nasal passages. A resilient means or spring member extends along the length of the truss member. The spring member, when the truss member is in place, acts to stabilize the outer wall tissue and thereby prevents the outer wall tissue of the first and second nasal passages from drawing in during breathing.

In one known nasal dilator, such as disclosed in U.S. Pat. No. 6,318,362, the spring member consists of a pair of resilient bands. The first resilient band is secured to run along the length of the nasal dilator. The second resilient band of the spring member is spaced from the first resilient band and also runs along the length of the nasal dilator. The first and second resilient bands are relatively stiff and are oriented generally parallel to one another and substantially parallel to the longitudinal extent of the nasal dilator. The resiliency of the first and second resilient bands prevents the outer wall tissue of the first and second nasal passages from drawing in during breathing.

In some of the known nasal dilators the truss member further includes an adhesive substance located on a second side of the flexible strip of material. The adhesive substance acts to releasably secure the truss member to the outer wall tissue of the first and second nasal passages. First and second release liners cover the adhesive substance on the second side of the flexible strip of base material. The first and second release liners are readily removable from the strip of base material to expose the adhesive substance and permit the truss member to be secured to the outer wall tissue of the first and second nasal passages.

FIG. 1 depicts a prior art nasal dilator in use and having a flexible base strip of material 18 adapted to be adhered to nasal tissue surfaces and a pair of resilient bands 30a, 30b. Additional details of this nasal dilator are disclosed in U.S. Pat. No. 5,533,503. Removal of the known nasal dilators is typically performed in a “side-to-side” or “side-to-center” manner wherein the direction of removal is generally aligned with a longitudinal direction of the nasal dilator. Nasal dilators, such as illustrated in FIG. 1, are typically removed by grasping the ends of the dilator and lifting the ends away from the nasal tissue surface and towards he nasal bridge. The resilient members are relatively wide (measured in a direction generally transverse to a longitudinal direction of a relaxed nasal dilator) and stiff so that the nasal dilator is most easily removed from the nasal surfaces in this end-to-center approach. During the removal a user's skin may be damaged as a relatively low peel angle of removal combined with the stiff resilient bands transfers high tensile forces to the skin surfaces during the removal process. FIG. 2 depicts an applied nasal dilator having a relatively low peel angle, a. The rigidity of the resilient bands results in the peel angle, a, as illustrated in FIG. 2, being typically less than 45°. The small peel angle for removing the nasal dilator results in a transferral of a large amount of the peel force directly to the skin of the user, leading to irritation or damage to nasal skin surfaces.

There exists a need in the art to provide a nasal dilator having a substantially greater peel angle during removal as compared to known nasal dilators.

### SUMMARY OF THE INVENTION

The present invention includes a nasal dilators with an improved spring element comprising multiple filaments. The nasal dilator has a resilient element, or “spring,” made up of a plurality of small filaments for keeping the nasal passages from drawing in while breathing. The filaments may be a variety of shapes and sizes and may run both along the length of the nasal dilator and at a variety of different angles relative to the length of the nasal dilator. The filaments further allow the nasal dilator of the present invention to be removed from the nose in a “top-to-bottom” fashion. The top-to-bottom peel method allows a greater peel angle and so results in less peel force being transferred to the skin of the use.

### DESCRIPTION OF THE DRAWINGS

FIG. 1 is a side elevational view of a nasal dilator of the prior art as placed on a nose.

FIG. 2 depicts removal of the nasal dilator of FIG. 1.

FIG. 3 is a side elevational view of a nasal dilator of the present invention as placed on a nose

FIG. 4 is a top view of an alternative embodiment nasal dilator of the present invention.

FIG. 5 is a top view of another alternative embodiment of the present invention.

FIG. 6 is a top view of another alternative embodiment of the present invention.

FIG. 7 is a perspective shadow view of a user removing the nasal dilator of the present invention.

FIG. 8 is a depiction of a nasal dilator of the present invention being removed from application and illustrating the peel angle.

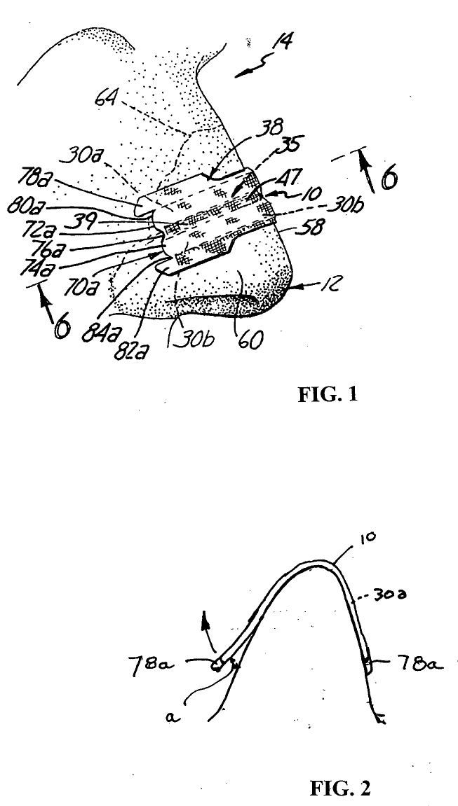

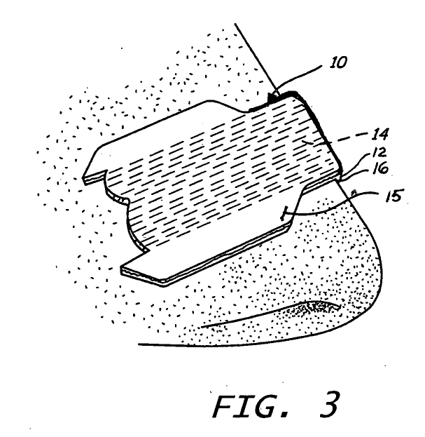

### DESCRIPTION OF THE INVENTION

As illustrated in FIGS. 3-8, the present invention includes a nasal dilator 10. The nasal dilator 10 defines a truss member including at least a flexible strip of base material 12, a resilient element 13. An adhesive 16 is used to secure the truss member to nasal surfaces of a user. The truss defines a first end region 20 and a second end region 22 coupled to the first end region by way of an intermediate segment 24. The flexible strip of base material 12 is preferably formed of an interwoven piece of fabric that allows the skin of he nose to breath to maximize comfort and minimize irritation. As an alternative, the strip of base material 12 may be formed of a plastic film. The truss member may also include a flexible strip of top material 15 so that the resilient element is disposed between top and bottom strips of material.

The truss member may be made of rubber, vinyl, cloth, soft plastic, or any other material known in the art to be pliable under the conditions for which the nasal dilator 10 is to be used. Those of ordinary skill in the art will recognize that the materials used to make the truss member must withstand the forces placed thereon and also withstand the foreign objects and materials that the nasal dilator 10 may come into contact with, including water, sweat, etc.

The resilient element 13 is fixedly attached or integrated within the truss member and may further include a plurality of filaments 14. The adhesive material 16 is placed on one side of the truss member 12 such that the nasal dilator 10 can be removably affixed to the nose of a user.

The adhesive material 16 is preferably a bio-compatible adhesive that is compatible with the skin of the nose but strong enough such that it can maintain the nasal dilator 10 in the correct position during use. A number of different types of adhesives are known to those in the art such as breathable, acrylic, pressure sensitive bio-compatible adhesives.

As previously mentioned, the resilient element 13 of the present invention includes filaments 14. Spring element 13 includes a plurality of individual filaments 14. The filaments 14 may be constructed of a variety of different materials, such as, for example, polymers, fiberglass, metal, glass fibers, or polymer coated glass fibers. The term “filament” is also not limited to long thing strands of uniform material. The term filament, rather, is intended to encompass a wide variety of different materials in different configurations, some of which are further discussed below.

In the embodiment of FIG. 3, the filaments 14 are adhesively secured within the truss member between the top and bottom strips of material 15, 12. In other embodiments of the present invention not having the top strip of material 14, the filaments 15 may be adhered to the bottom strip of material 12.

The filaments 14 may be provided in a single layer or multiple layers. The important factor in determining the composition, shape, and size of the filament 14 is that, when placed on the nose such that the nasal dilator 10 is bent in a substantially “U-shape,” the nasal dilator 10 can be peeled off in a top-to-bottom fashion because the localized rigidity of the truss member as measured perpendicular to its long axis is substantially reduced, as compared to the prior art nasal dilators, due to each individual filament 14 being less rigid in a transverse direction than the resilient bands of the prior art. The spring effect of the aggregate of all of the filaments 14 along the long axis of the truss member 12, however, is at least comparable to the spring effect of the resilient bands of the prior art nasal dilators.

The resilient element 13 imparts upon the truss member a force similar to the prior art BREATH RIGHT brand nasal strips. The nasal dilator 10 therefore imparts upon the nasal passages a force sufficient to prevent the nasal passage from drawing inwards during breathing. As discussed further below, however, the filaments 14 that form the spring element 13 allow for a substantially easier and more comfortable removal of the nasal dilator 10 after use.

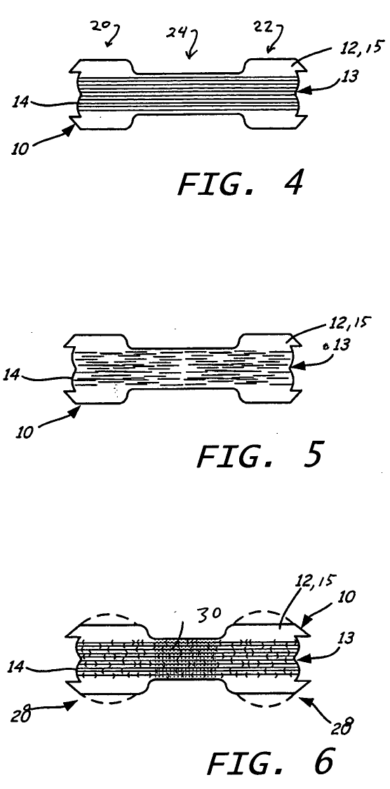

As shown in FIG. 4, in one embodiment the filaments 14 run substantially the full length of the nasal dilator 10. In this embodiment a plurality of spaced individual filaments 14 make up the spring element 13. Each filament 14 extends generally the entire length of the truss member. The filaments 14 may be generally evenly spaced from one another and may be generally parallel to the longitudinal axis of the truss member.

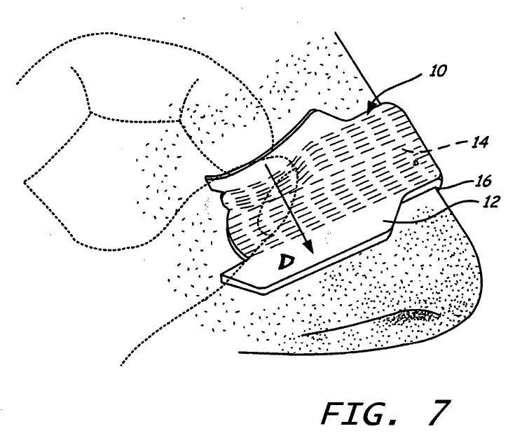

In another embodiment of the present invention as illustrated in FIG. 5, the filaments 14 could be shorter than the length of the truss member. In another embodiment (not shown), a combination of short and long and stiff and soft filaments 14 may be used to form the spring element 13 and to provide the desired spring force to the truss member. In such a design the filaments 14 may or may not overlap a line perpendicular to the longitudinal direction of truss member 10. The filaments 14 could be in single or multiple layers.

In further embodiments (not shown), some or all filaments 14 may be provided at other angles relative to the longitudinal direction of truss member 12. Providing filaments 14 at varying angles relative to the longitudinal direction of the truss member may provide a better stabilization of the outer wall tissue of the nose and result in better prevention of the passages from drawing in during breathing.

The filaments 14 may also form a woven structure to form the spring element 13, such as illustrated in FIG. 6. Stiff and/or spring-like filaments 14 may be woven together with more relaxed filaments 14 to form a woven structure for the resilient element 13. Such a resilient element 13 may have a different spring constant depending on the direction of bending. In other words, the spring constant could be greater when the nasal dilator 10 is bent about an axis other than its longitudinal axis.

Filaments 14 may be cylindrical in form, such as a circular cylinder, an elliptical cylinder, a triangular cylinder, etc. Preferably, resilient element 13 of nasal dilator 10 includes five or more individual filaments 14.

The removal of the nasal dilator 10 of the present invention is depicted in FIG. 7. The nasal dilator 10 is depicted as being removed in a “top-to-bottom” manner according to the invention. A direction of removal, which is generally transverse to the longitudinal axis of the nasal dilator 10, is indicated by arrow D. The filaments 14 of the nasal dilator 10 allow the nasal dilator 10 to be removed in a substantially easier manner than the prior art devices. The removal direction is substantially from top-to-bottom, i.e., perpendicular to the length of the nasal dilator 10. Peeling the nasal dilator 10 in such a manner allows the peel angle to be maximized closer to 180° such that that there is less potential for skin damage.

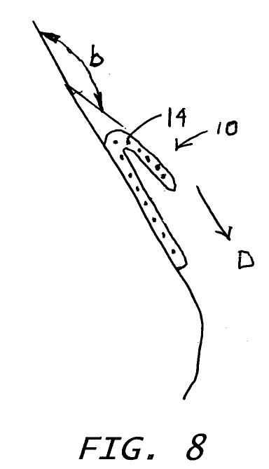

The filaments 14 of the nasal dilator 10 are individually small and pliable enough such that the nasal dilator 10 can be removed in the “top-to-bottom” direction without imparting unnecessary force directly to the skin of the user. Ideally, to minimize skin damage, the peel angle of the adhesive 16 from the skin of the user should be close to 180°. Preferably the peel angle is greater than 120° and more preferably greater than 150°. FIG. 8 depicts removal of the nasal dilator 10 in a “top-to-bottom” manner according to the invention. The peel angle is designated as “b” in FIG. 8.

Other changes to a nasal dilator 10 may also enhance the ease of removal of the nasal dilator 10 in a “top-to-bottom” fashion. For example, as illustrated in FIG. 6, the tab area 28 of the nasal dilator 10 could be enlarged. Enlarging the tab area 20 of the nasal dilator 10 provides the user with a larger piece of the dilator 10 to grasp during removal. FIG. 4 illustrates in phantom lines the tabs 28 being enlarged and rounded. This also provides a more convenient region for the user to grasp the nasal dilator 10 during removal and also helps to center the user's force of removal. An adhesive void 30 over the bridge of the nose allows the removal forces on the two sides of the nose to act independently and, at the same time, may reduce the total amount of removal force necessary to remove the nasal dilator 10.

The embodiments described herein are for illustrative purposes and are not meant to exclude any derivations or alternative methods that are within the conceptual context of the invention. It is contemplated that various deviations can be made to these embodiments without deviating from the scope of the present invention. Accordingly, it is intended that the scope of the present invention be dictated by the appended claims rather than by the foregoing description of this embodiment.

1. A nasal dilator for preventing outer wall tissue of nasal passages of a nose from drawing in during breathing comprising:
a flexible truss member including:
a flexible strip of material defining first and second end regions and an intermediate segment with the first end region adapted to engage the outer wall tissue of a first nasal passage at a first side of the flexible strip of material and with the second end region adapted to engage the outer wall tissue of a second nasal passage at the first side of the flexible strip of material, the intermediate segment configured to traverse a portion of a user's name located between the first and second nasal passages, the tendency of the truss member to return to its initial state when flexed acting to stabilize the outer wall tissue and thereby prevent the outer wall tissue of the first and second nasal passages from drawing in during breathing; and
a resilient element having a plurality of filaments and capable, at least in part, of resilient deformation to allow the truss member to conform to the outer wall tissues of the first and second nasal passages and to provide said truss member with said tendency to return to its initial state when flexed.

2. The nasal dilator of claim 1, and further including:
an adhesive substance located on the first side of the flexible strip of material at the first and second end regions thereof, so as together with the resilient element being capable, at least in part, of resilient deformation, the adhesive substance for releasably securing the truss member to the outer wall tissues of the first and second nasal passages.

3. The nasal dilator of claim 1 wherein the flexible strip of material is formed of a piece of fabric.

4. The nasal dilator of claim 1 wherein the resilient element includes:
a plurality of filaments which are oriented substantially parallel to a longitudinal extent of the flexible strip of material.

5. The nasal dilator of claim 1 wherein the resilient element includes a plurality of cylindrical filaments.

6. The nasal dilator of claim 1 wherein the resilient element includes a plurality of filaments which extend across generally an entire length of the nasal dilator.

7. The nasal dilator of claim 1 wherein the resilient element are provided in a separated side-by-side manner.

8. The nasal dilator of claim 1 wherein the resilient element includes a plurality of filaments which are substantially shorter than an entire length of the nasal dilator.

9. The nasal dilator of claim 8 wherein the plurality of filaments are provided in an overlapping relationship.

10. The nasal dilator of claim 1 wherein the plurality of filaments include between 5 to 10 individual filaments.

11. The nasal dilator of claim 1 wherein the plurality of filaments includes at least 10 individual filaments.

12. A nasal dilator for preventing outer wall tissue of nasal passages of a user's nose from drawing in during breathing, comprising:
a flexible truss member having an initial state absent flexure thereof, the truss member including:
a first end region with an end region surface having an adhesive thereat so as to be adapted to adhesively engage the outer wall tissue of a first nasal passage;
a second end region with an end region surface having an adhesive thereat so as to be adapted to adhesively engage the outer wall tissues of a second nasal passage;
an intermediate segment configured to traverse a portion of the user's nose located between the first and second nasal passages; and
a resilient member having a plurality of filaments in at least a portion of the first and second end regions and the intermediate segment, the resilient member being capable, at least in part, of resilient deformation that tends to cause the first and second end regions to separate from one another after being urged toward one another to give the truss member a tendency to return to its initial state when flexed to thereby act to stabilize the outer wall tissue if engaged therewith and so prevent the outer wall tissues of the first and second nasal passages from drawing in during breathing.

13. The nasal dilator of claim 12, and further including:
an adhesive substance located on the first side of the flexible strip of material at the first and second end regions thereof, so as together with the resilient element being capable, at least in part, of resilient deformation, the adhesive substance for releasably securing the truss member to the outer wall tissues of the first and second nasal passages.

14. The nasal dilator of claim 12 wherein the resilient element includes:
a plurality of filaments which are oriented substantially parallel to a longitudinal extent of the flexible strip of material.

15. The nasal dilator of claim 12 wherein the resilient element includes a plurality of cylindrical filaments.

16. The nasal dilator of claim 12 wherein the resilient element includes a plurality of filaments which extend across generally an entire length of the nasal dilator.

17. The nasal dilator of claim 12 wherein the resilient element includes a plurality of filaments which are substantially shorter than an entire length of the nasal dilator.

18. The nasal dilator of claim 17 wherein the plurality of filaments are provided in an overlapping relationship.

18. The nasal dilator of claim 12 wherein the plurality of filaments include between 5 to 10 individual filaments.

19. The nasal dilator of claim 12 wherein the plurality of filaments includes at least 10 individual filaments.

20. A method of using a nasal dilator comprising
providing a nasal dilator having a resilient member defined by a plurality of filaments in at least a portion of first and second end regions and an intermediate segment, the resilient member being capable, at least in part, of resilient deformation that tends to cause the first and second end regions to separate from one another after being urged toward one another to give the nasal dilator a tendency to return to its initial state when flexed to thereby act to stabilize outer wall tissue if engaged therewith and so prevent the outer wall tissues of first and second nasal passages from drawing in during breathing;
applying the nasal dilator to a nasal skin surface;
using the nasal dilator for a period of time; and
removing the nasal dilator from the nasal skin surface by peeling the nasal dilator in a top-to-bottom direction.

21. A method of using a nasal dilator that can be removed using a high peel angle, comprising:
forming a nasal dilator having a plurality of resilient filaments which together bias the nasal dilator into a normally flat planar shape;
applying the nasal dilator to a user; and
removing the nasal dilator by peeling the nasal dilator in a top-to-bottom fashion utilizing a high peel angle.

22. The method of using a nasal dilator of claim 21 wherein the high peel angle is approximately 150° or greater.

23. The method of using a nasal dilator of claim 21 wherein the high peel angle is approximately 120° or greater.
